(19) United States
(12) Patent Application Publication
(10) Pub. No.: US 2024/0066038 A1
(43) Pub. Date: Feb. 29, 2024

(54) WNT MODULATORS TO TREAT CENTRAL NERVOUS SYSTEM INJURIES

(71) Applicant: MAYO FOUNDATION FOR MEDICAL EDUCATION AND RESEARCH, Rochester, MN (US)

(72) Inventors: Guojun Bu, Jacksonville, FL (US); Yonghe Li, Ponte Vedra Beach, FL (US); Thomas Caulfield, Jacksonville, FL (US)

(21) Appl. No.: 18/260,423

(22) PCT Filed: Jan. 6, 2022
(86) PCT No.: PCT/US22/11456

§ 371 Date: Jul. 5, 2023

Related U.S. Application Data
(60) Provisional application No. 63/134,351, filed on Jan. 6, 2021.

(51) Int. Cl. A61K 31/55; A61K 31/397; A61K 31/40; A61K 31/438; A61K 31/44; A61K 31/4427; A61K 31/4439; A61K 31/445; A61K 31/4545; A61K 31/4709; A61K 31/497; A61K 31/501; A61P 25/28

(52) U.S. Cl. CPC A61K 31/55; A61K 31/397; A61K 31/40; A61K 31/438; A61K 31/44; A61K 31/4427; A61K 31/4439; A61K 31/445; A61K 31/4545; A61K 31/4709; A61K 31/497; A61K 31/501; A61P 25/28

(57) ABSTRACT

Provided herein are methods of treating a central nervous system injury by administering a Wnt modulator to a subject suffering therefrom.

### CROSS-REFERENCE TO RELATED APPLICATIONS

This application claims the benefit of priority to U.S. Provisional Patent Application No. 63/134351 filed Jan. 6, 2021, the disclosure of which is incorporated herein by reference in its entirety.

### BACKGROUND

Wnt proteins are secreted glycoproteins which bind to cell surface receptors to activate β-catenin-dependent (canonical) and -independent (non-canonical) signaling pathways. A hallmark of the activation of Wnt/β-catenin signaling is the stabilization of cytosolic β-catenin, which enters the nucleus and subsequently binds to transcription factors of the T cell factor/lymphoid enhancing factor (TCF/LEF) family to induce the expression of specific target genes. In the absence of Wnt ligands, β-catenin is phosphorylated by a supramolecular complex containing adenomatous polyposis coli (APC), axin, and glycogen synthetase kinase 3β (GSK3β), and phosphorylated β-catenin becomes multi-ubiquitinated (Ub) and is degraded by the 26S proteasome. However, when Wnt proteins interact with both LRP6 and Fzd, signaling from the cell surface proceeds through the proteins dishevelled (Dvl), axin and GSK3β, resulting in the phosphorylation of LRP6 cytoplasmic tail, the inhibition of GSK3β and the stabilization of cytosolic β-catenin stabilization [1, 2].

In the brain, Wnt/β-catenin signaling is not only crucial for neuronal survival and neurogenesis, but it plays important roles in regulating synaptic plasticity, neuroinflammation, angiogenesis and blood-brain barrier integrity and function [3-5]. While the Wnt/β-catenin pathway is tightly regulated in the adult brain to maintain neurovascular integrity and CNS functions, it is frequently dysregulated in CNS injuries [3-5]. Therefore, restoring Wnt/βcatenin signaling represents a unique opportunity for the rational design of treatment for CNS injuries.

### SUMMARY

Provided herein are compounds that can modulate Wnt, and can be used to treat central nervous system injuries. In particular, compounds for use in the disclosed methods are as disclosed in Tables A or B, or have a structure of Formula (I) or (I′):

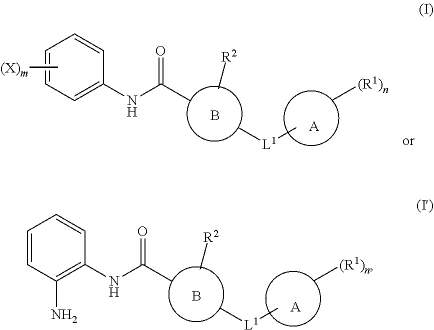

wherein ring B-R² is

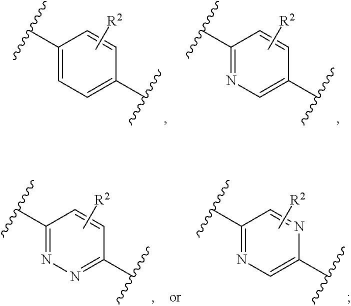

L¹ is NH—CO—C₀₋₃alkylene or CO—NH—C₀₋₄alkylene; ring A is a 4-12-membered monocyclic, bicyclic, bridged, or spiro heterocycle comprising a nitrogen ring atom; each R¹ is independently H, C₁₋₆alkyl, halo, C₁₋₆haloalkyl, C₁₋₃alkylene-O—C₀₋₃alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl; R² is H, F, OH, OMe, or NH₂; each X is independently NH₂, NMe₂, F, or CF₃, m is 1 or 2, and n is 1, 2, or 3, with the proviso that when ring A comprises piperidinyl, at least one R¹ is other than H.

In some cases, the CNS injury is a stroke, or more specifically, an ischemic stroke or a hemorrhagic stroke. In some cases, the CNS injury is a traumatic brain injury. In some cases, the CNS injury is a spinal cord injury. In some cases, the CNS injury is a cerebral small vessel disease.

### BRIEF DESCRIPTION OF THE FIGURES

FIG. 1 shows that Wnt activators activate Wnt/β-catenin signaling in neuronal N2a cells. (A) Compound 1039 activates Wnt/β-catenin signaling in HEK293 cells. HEK293 cells were transfected with Super8XTOPFlash and 8-galactosidase vectors along with Wnt3A or control vector. After 24 h incubation, cells were treated with 1039 at the indicated concentrations for 24 h. The luciferase activity was then measured and normalized to the activity of the 8-galactosidase. All the values are the average of triplicate determinations with the SD values indicated by error bars. This experiment is a representative of 3 such experiments performed with similar data. (B, C) N2a cells were treated with Compound 1039 at the indicated concentrations for 24 h. (B) The levels of total cellular β-catenin were examined by Western blotting. (C) The intensity of the β-catenin bands was quantified and normalized to the corresponding signal for actin. The results represented in the histograms are shown as the mean±SD and are the average of 3 independent experiments.

FIG. 2 shows Compound 1015 activates Wnt/β-catenin signaling in hCMEC/D3 human cerebral microvascular endothelial cells. hCMEC/D3 cells were treated with Compound 1015 at the indicated concentrations in serum free medium for 24 h. The expression level of Axin2, a specific target of Wnt/β-catenin signaling, was measured by qPCR. The results represented in the histograms are shown as the means±SE and are the average of 3 independent experiments. One-way ANOVA with post hoc Tukey-Kramer honest significant difference test. **, p<0.01.

### DETAILED DESCRIPTION

Provided herein are methods of treating a central nervous system (CNS) injury by administering to a subject suffering therefrom a Wnt modulator as disclosed herein. In brain, Wnt/β-catenin signaling is not only crucial for neuronal survival and neurogenesis, but it plays important roles in regulating synaptic plasticity, neuroinflammation, angiogenesis and blood-brain barrier integrity and function [3-5]. While the Wnt/β-catenin pathway is tightly regulated in the adult brain to maintain neurovascular integrity and CNS functions, it is frequently dysregulated in CNS injuries [3-5]. Therefore, restoring Wnt/βcatenin signaling represents a unique opportunity for the rational design of treatment for CNS injuries.

Stroke: Stroke is the No. 5 cause of death and a leading cause of long-term disability in adults in the United States. Early management of the poststroke complications in acute stroke patients is critical for preventing further brain damage or promoting repair. Due to the time-limited treatment window, thrombolytic therapy with tissue plasminogen activator (tPA), the only FDA approved pharmacological intervention, is limited to 3%-5% of acute stroke patients. Accumulating evidence suggests that substantial functional improvement after stroke can be achieved with subacutely pharmacological therapies [6].

Ischemic stroke, which makes up about 85% of stroke, occurs when the cerebral blood flow (CBF) is abruptly blocked by an embolus or thrombus. Mounting evidence indicates that activation of Wnt/β-catenin signaling may prevent, ameliorate, or even reverse the negative effects of ischemic brain injury [4, 5, 7]. Indeed, up-regulation of Dkk1, an antagonist of the Wnt/β-catenin pathway by binding to the Wnt co-receptor LRP6, and downregulation of Wnt/β-catenin signaling were detected within the ischemic region as early as 3 h after middle artery occlusion (MCAo) [8]. Moreover, Dkk1 level is significantly increased in peripheral blood of patients with acute ischemic stroke or with stable cerebrovascular disease [9, 10]. Importantly, intranasal Wnt3A displayed significantly neuroprotective and regenerative effects after focal ischemic stroke in mice and rats [11, 12]. Interestingly, most of the beneficial effects of Wnt3a were abolished after intranasal administration of Wnt antagonist Dkk1 or small molecule Wnt inhibitors XAV-939 [11]. Together, these studies indicate that the Wnt/β-catenin pathway is an attractive therapeutic target to promote neurovascular repair following ischemic stroke.

Hemorrhagic stroke, which makes up about 15% of stroke cases, is a devastating pathological condition as it is likely to cause fatality or severe disability in survivors. Studies have demonstrated that Wnt/β-catenin signaling plays a critical role in disease pathogenesis of hemorrhagic stroke [4, 7]. Particularly, the Wnt/β-catenin signaling pathway is greatly inhibited as early as 6 h after onset of hemorrhagic stroke [13, 14], and remains downregulated up to 2 weeks after onset [4]. Importantly, exogenous intranasal delivery of Wnt3A into the rat brain with subarachnoid hemorrhage (SAH) alleviated neuronal apoptosis, improved the neurological scores, brain water content and long-term neurobehavioral functions after SAH, and these beneficial effects was totally abolished after administration of Fzd-1 siRNA [14]. Similarly, exogenous delivery of recombinant human Wnt1 into the ventricle of SAH rats alleviated subarachnoid hemorrhage-induced early brain injury, which was abolished after administration of Wnt1 siRNA or a neutralizing monoclonal antibody anti-

Fzd1 [13]. Together, these studies indicate that the Wnt/β-catenin pathway is an attractive therapeutic target to promote neurovascular repair following hemorrhagic stroke.

Traumatic brain injury: Traumatic brain injury (TBI) is an injury to the brain caused by a physical force, leading to temporary or permanent impairment in cognitive, physical, and psychosocial functions [15]. Numerous studies have demonstrated that Wnt/β-catenin signaling plays an important role in TBI pathobiology and is a novel target for intervention in secondary injury after TBI [4, 7]. It has been reported that Dkk1 level is elevated in the serum of patients with severe TBI, and is closely associated with increasing severity of the trauma and higher risk of short-term mortality [16]. Moreover, Wnt3A given via intranasal administration or intravenous injection improves functional recovery after TBI by modulating autophagic, apoptotic, and regenerative pathways in the mouse brain [17, 18]. Therefore, Wnt/β-catenin signaling plays an important role in TBI pathobiology, and this pathway is a novel target for intervention in secondary injury after TBI [4, 7].

Spinal cord injury: Spinal cord injury (SCI) is a sudden onset disruption to the neuronal tissue within the spinal canal and can be classified into primary and secondary injuries. While the primary injury is an irreversible damage caused by the initial traumatic event, the secondary injury is created by a series of biological and functional changes including spinal cord hemorrhage, edema, ischemia reperfusion, apoptosis, inflammatory reaction, etc [19]. Although Wnts are barely expressed in uninjured spinal cord, exogenous Wnt3a administration is able to promote axon conduction and regeneration, reduce neuronal cell death, and ultimately enhance the recovery of neurological function after SCI [20-22].

Cerebral small vessel disease: Cerebral small vessel disease (CSVD) is refers to different pathological processes that affect the small vessels of the brain, including small arteries, arterioles and capillaries, and can cause stroke and dementia, mood disturbance and gait problems [23, 24]. Blood-brain barrier (BBB) damage is a critical pathological feature of CSVD [24]. Studies in past ten years have established that the Wnt/β-catenin pathway is a key pathway required not only for BBB formation but also for BBB integrity and function [3, 24-26]. Indeed, dysregulation of Wnt/β-catenin may limit remyelination of white matter lesions (WMLs) and render the BBB leaky [24, 27, 28]. Therefore, restoring Wnt/β-catenin signaling brings therapeutic opportunities to repair BBB damages and reduce white matter injuries in CSVD.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound as disclosed herein to an individual in need of such treatment.

The term “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, reduction in symptoms, or by any of the assays or clinical diagnostic tests described herein or known in the art. The precise effective amount for a subject will depend upon the subjects body weight, size, and health; the nature and extent of the condition; and the therapeutic or combination of therapeutics selected for administration. Therapeutically effective amounts for a given situation can be determined by routine experimentation that is within the skill and judgment of the clinician.

Presented herein are methods of administering a compound as disclosed herein or salt thereof as the neat compound or as a pharmaceutical composition orally, intravenously, or parenterally. In some cases, the compound or salt thereof is administered orally. Administration of a pharmaceutical composition, or neat compound, can be performed during or after the onset of the disease or condition of interest. Typically, the pharmaceutical compositions are sterile, and contain no toxic, carcinogenic, or mutagenic compounds that would cause an adverse reaction when administered.

### Compounds for Use in the Disclosed Methods

Provided herein are methods of treating CNS injuries by administering a Wnt modulator to a subject suffering therefrom. The Wnt modulator is a compound as discussed below, or a pharmaceutically acceptable salt thereof. The compounds can have a structure of formula (I), or more specifically (I′), or pharmaceutically acceptable salts thereof:

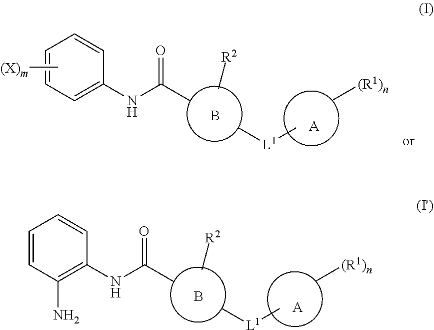

wherein
ring B-R² is

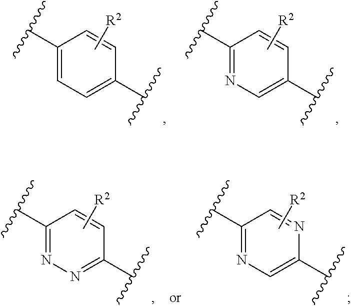

L¹ is NH—COC₀₋₃alkylene or CO—NH—C₀₋₄alkylene;
ring A is a 4-12-membered monocyclic, bicyclic, bridged, or spiro heterocycle comprising a nitrogen ring atom;
each R¹ is independently H, C₁₋₆alkyl, halo, C₁₋₆haloalkyl, C₁₋₃alkylene-O—C₁₋₃alkyl, C₀₋₃alkylene-C₃-C₈carbocycle, C₀₋₃alkylene-C₃-C₈-membered heterocycle, C₀₋₃alkylene-5-7-membered heteroaryl, or C₀₋₃alkylene-C₆₋₁₀aryl;
R² is H, F, OH, OMe, or NH₂;
each X is independently NH₂, NMe₂, F, or CF₃;
n is 1, 2, or 3; and
m is 1 or 2,
   with the proviso that when ring A comprises piperidinyl, at least one R¹ is other than H.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁₋₆ alkyl refers to an alkyl group having a number of carbon atoms encompassing the entire range (e.g., 1 to 6 carbon atoms), as well as all subgroups (e.g., 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. A “haloalkyl” group is an alkyl group having at least one halo substituent. In some cases, the haloalkyl comprises 1, 2, or 3 halo substituents, or can comprise a perhaloalkyl (i.e., all hydrogen atoms of the alkyl group are substituted with a halo). Non-limiting examples of haloalkyl include trifluoromethyl, fluoroethyl, difluoroethyl, and trifluoroethyl.

The term “alkylene” used herein refers to an alkyl group having a substituent. For example, an alkylene group can be —CH₂CH₂— or —CH₂— or —CH₂CH(CH₃)—. The term Cₙ means the alkylene group has “n” carbon atoms. For example, C₁₋₄ alkylene refers to an alkylene group having a number of carbon atoms encompassing the entire range, as well as all subgroups, as previously described for “alkyl” groups. A C₀alkylene group refers to a direct bond. Unless otherwise indicated, an alkylene group can be an unsubstituted alkylene group or a substituted alkylene group.

As used herein, the term “carbocycle” refers to an aliphatic cyclic hydrocarbon group containing three to eight carbon atoms (e.g., 3, 4, 5, 6, 7, or 8 carbon atoms). The term Cₙ means the carbocycle group has “n” carbon atoms. For example, C₅ carbocycle refers to a carbocycle group that has 5 carbon atoms in the ring. C₆-C₈ carbocycle refers to carbocycle groups having a number of carbon atoms encompassing the entire range (e.g., 6 to 8 carbon atoms), as well as all subgroups (e.g., 6-7, 7-8, 6, 7, and 8 carbon atoms). Nonlimiting examples of carbocycle groups include cyclopropyl, cyclobutyl, cyclopentyl, cyclohexyl, cycloheptyl, and cyclooctyl. Unless otherwise indicated, a carbocycle group can be an unsubstituted carbocycle group or a substituted carbocycle group.

As used herein, the term “heterocycle” is defined similarly as carbocycle, except the ring contains one to three heteroatoms independently selected from oxygen, nitrogen, and sulfur. In particular, the term “heterocycle” refers to a ring containing a total of three to twelve atoms (e.g., 3-8, 5-8, 3-6, 4-12, 3, 4, 5, 6, 7, 8, 9, 10, 11, or 12), of which 1, 2, or 3 of the ring atoms are heteroatoms independently selected from the group consisting of oxygen, nitrogen, and sulfur, and the remaining atoms in the ring are carbon atoms. The heterocycle can be monocyclic, bicyclic, bridged, or spiro heterocycle. In some cases, the heterocycle can be a 4-12 membered ring and comprises only one ring heteroatom—and in particular embodiments, a sole nitrogen ring heteroatom. Nonlimiting examples of heterocycle groups include piperdine, pyrazolidine, tetrahydrofuran, tetrahydropyran, dihydrofuran, morpholine, and the like. In some cases, the heterocycle can be azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl.

Carbocycle and heterocycle groups can be saturated or partially unsaturated ring systems optionally substituted with, for example, an R¹ group as disclosed herein. Heterocycle groups optionally can be further N-substituted with an R¹ group, e.g., alkyl (for example, methyl or ethyl), alkylene-carbocycle, alkylene-aryl, and alkylene-heteroaryl.

As used herein, the term “aryl” refers to a monocyclic or bicyclic aromatic group, having 6 to 10 ring atoms. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Aryl groups can be isolated (e.g., phenyl) or fused to another aryl group (e.g., naphthyl), a carbocycle group (e.g. tetraydronaphthyl), a heterocycloalkyl group, and/or a heteroaryl group.

As used herein, the term “heteroaryl” refers to a monocyclic or bicyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. In some cases, the heteroaryl is a 5-7 membered monocyclic ring having 1 to four ring heteroatoms. In some cases, the heteroaryl group is substituted with one or more of alkyl and alkoxy groups. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

In various cases, m is 1. In some cases, m is 1 and X is NH₂; and in some specific cases, X is meta to the amide bond on the phenyl ring, and in other specific cases, X is para to the amide bond on the phenyl ring. In some cases, m is 1 and X is NMe₂, and in some specific cases, X is ortho to the amide bond on the phenyl ring. In some cases, m is 1 and X is CF₃, and in some specific cases, X is meta to the amide bond on the phenyl ring. In some cases, m is 1, and X is F, and in some specific cases, X is ortho to the amide bond on the phenyl ring. In some cases, m is 2. In various cases, m is 2 and one X is NH₂and one X is F, and in some specific cases, each X is ortho to the amide bond on the phenyl ring.

In various cases, ring A of Formula (I) or (I′) can comprise azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl. In some cases, the substituent on ring A, R¹, can be fluoro, cyclopropyl, cyclopentyl, cyclohexyl, cycloheptyl, CH₂cyclohexyl, CH₂cyclopenyl, methyl, CH₂CH₂OCH₃, isopropyl, difluoroethyl, trifluoroethyl, tetrahydropyranyl, CH₂-(methyl-isoxazolyl), methyl-pyrazolyl, CH₂CH₂phenyl, CH₂phenyl, CH₂(methoxyphenyl), or phenyl.

In various cases, ring B-R² is

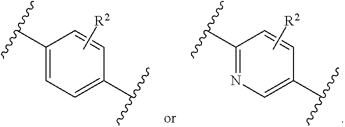

In various cases, ring B-R² is

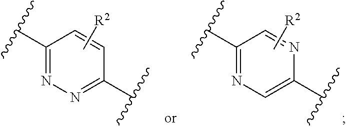

In some cases, R² is H. In some cases, R² is F. In some cases, R² is OH, OMe, or NH₂.

In various cases, L¹ is NHCO—C₀₋₄aklylene. In various cases, L¹ is CONH—C₀₋₄aklylene. In some cases, L¹ is CONHCH₂, or CONHCH₂CH₂, or CONHCH₂CH(CH₃).

In various cases, the compound can have a structure of Formula (IA), (IA′), (IA″), (IA′″), (IB), (IB′), (IC), or (IC′):

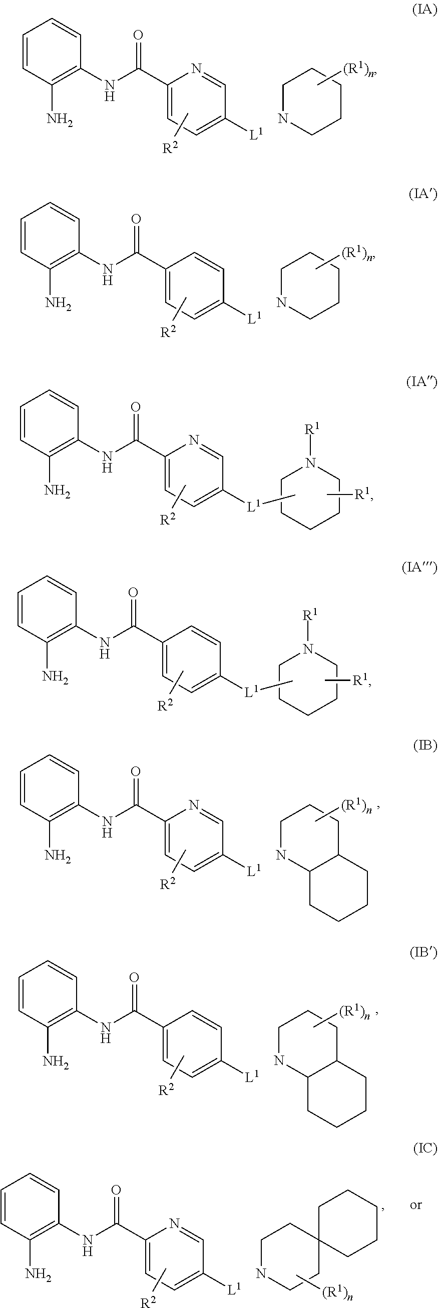

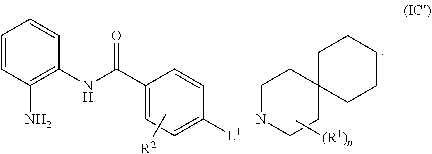

In some cases, the compound has a structure of Formula (ID) or (ID′):

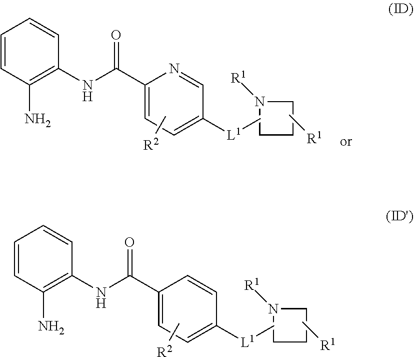

wherein when L¹ is attached to the ring nitrogen, R¹ on the ring nitrogen is null. In various cases, the compound has a structure of Formula (IE) or (IE′):

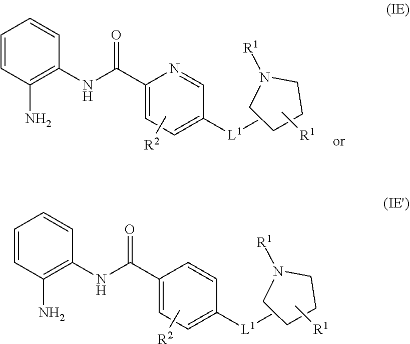

wherein when L¹ is attached to the ring nitrogen, R¹ on the ring nitrogen is null.

Specific compounds contemplated for use in the present disclosure include those as provided in Table A, below, or a pharmaceutically acceptable salt thereof.

TABLE A

| Structure | Compound |
|---|---|
| 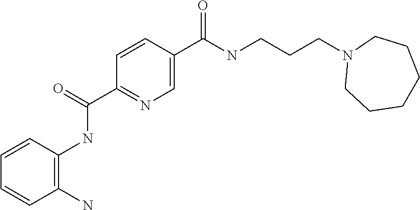 | 1001 |
| 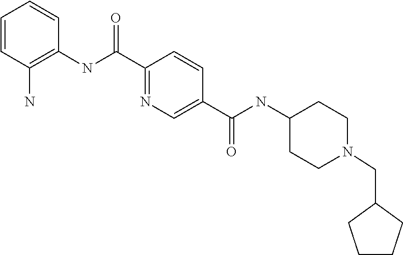 | 1002 |
| 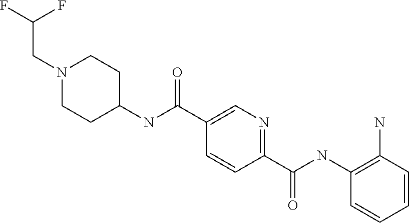 | 1003 |
| 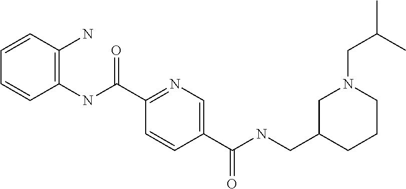 [SMILES: CC(C)CN1CCCC(CNC(=O)c2ccc(C(=O)Nc3ccccc3N)nc2)C1] | 1004 |
| 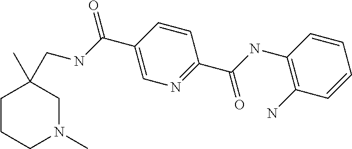 [SMILES: CN1CCCC(C)(CNC(=O)c2ccc(C(=O)Nc3ccccc3N)nc2)C1] | 1005 |
| 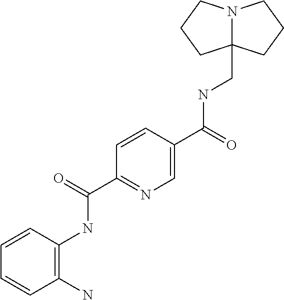 [SMILES: Nc1ccccc1NC(=O)c1ccc(C(=O)NCC23CCCN2CCC3)cn1] | 1006 |
| 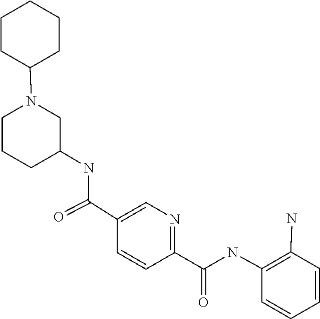 | 1007 |
| 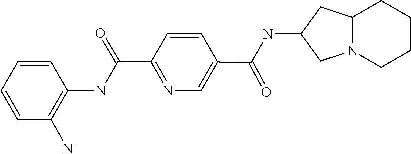 | 1008 |
| 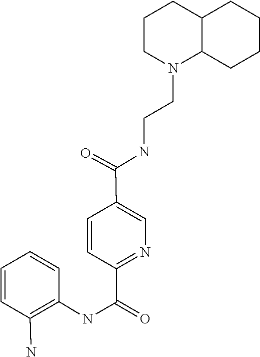 | 1009 |
| 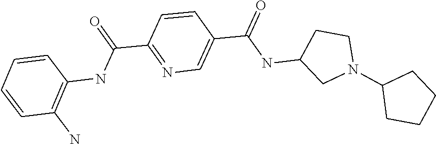 [SMILES: Nc1ccccc1NC(=O)c1ccc(C(=O)NC2CCN(C3CCCC3)C2)cn1] | 1010 |
| 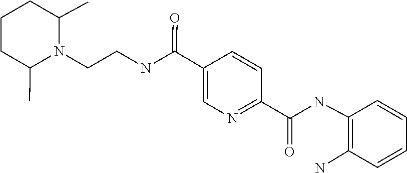 | 1011 |
| 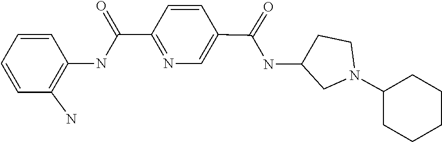 | 1012 |
| 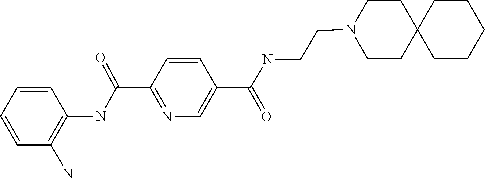 | 1013 |
| 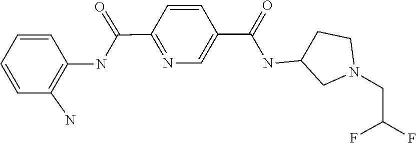 | 1014 |
| 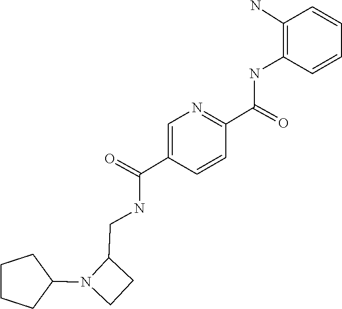 | 1015 |
| 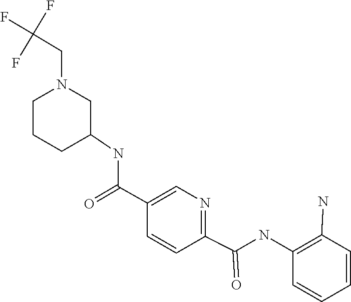 | 1016 |
| 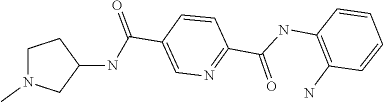 | 1017 |
| 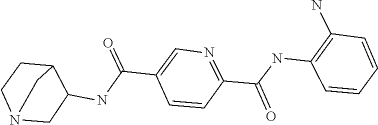 | 1018 |
| 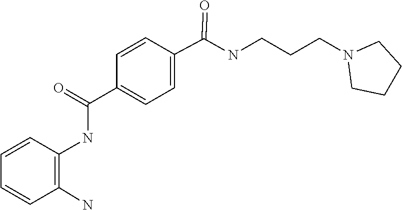 | 1019 |
| 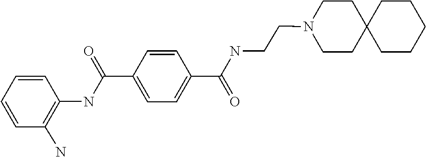 | 1020 |
| 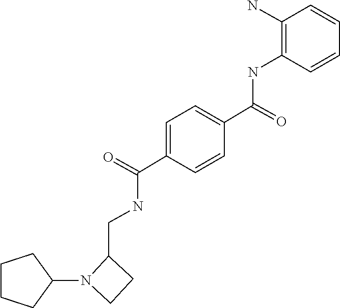 | 1021 |
| 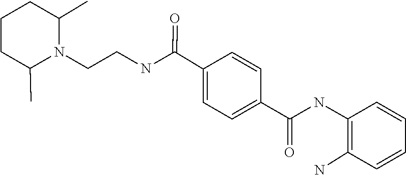 | 1022 |
| 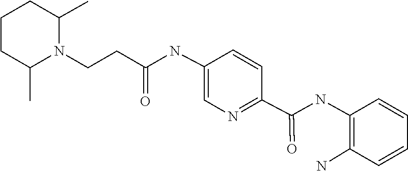 | 1023 |
| 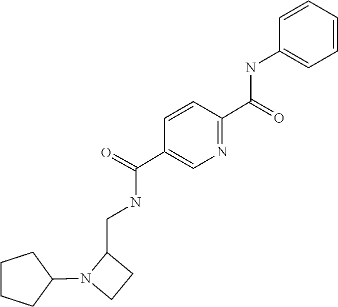 | 1024 |
| 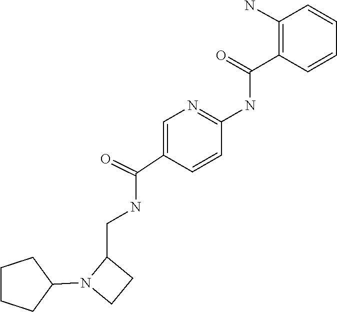 | 1025 |
| 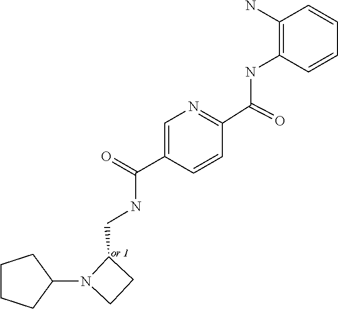 | 1026 |
| 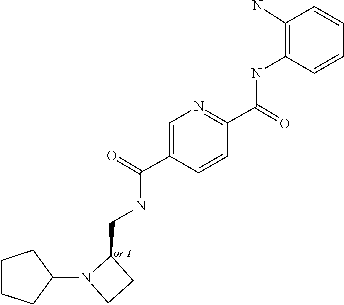 | 1027 |
| 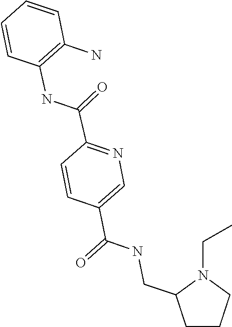 | 1028 |
| 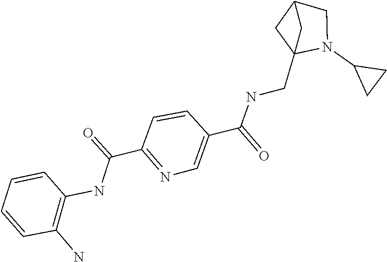 | 1029 |
| 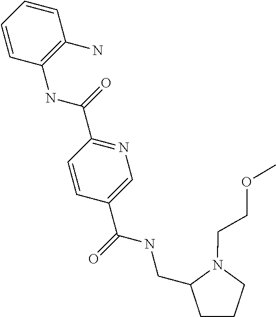 [SMILES: COCCN1CCCC1CNC(=O)c1ccc(C(=O)Nc2ccccc2N)nc1] | 1030 |
| 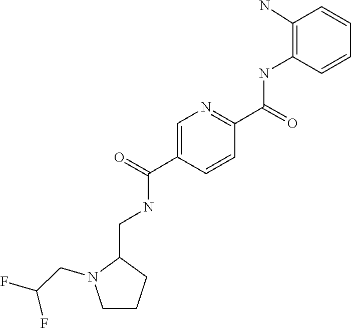 | 1031 |
| 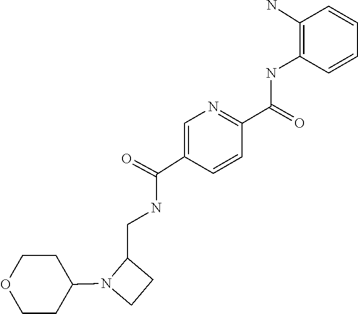 | 1032 |
| 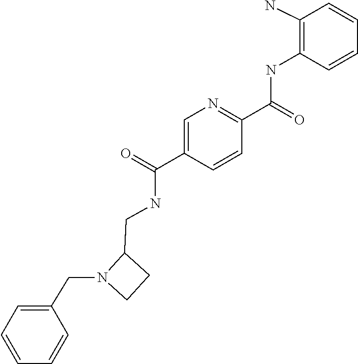 | 1033 |
| 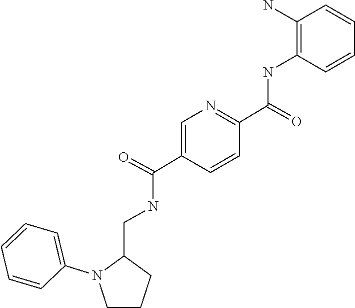 | 1034 |
| 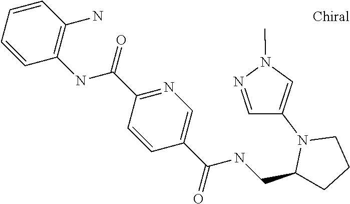 [SMILES: Cn1cc(N2CCC[C@H]2CNC(=O)c2ccc(C(=O)Nc3ccccc3N)nc2)cn1 ; "Chiral"] | 1035 |
| 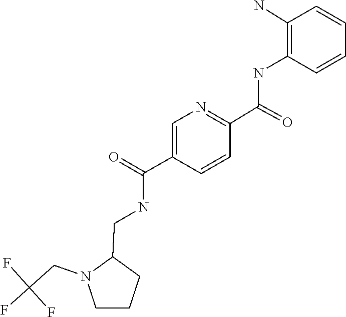 | 1036 |
| 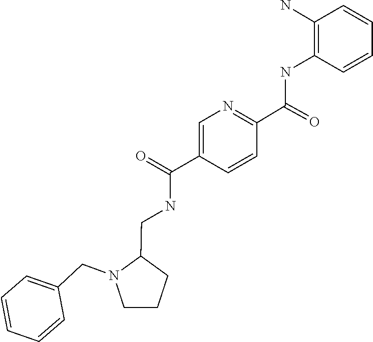 | 1037 |
| 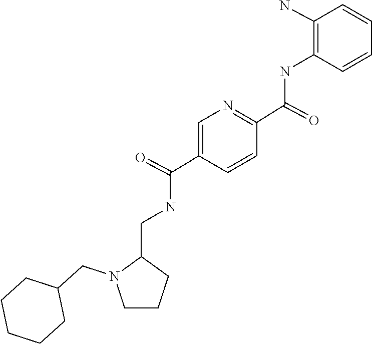 | 1038 |
| 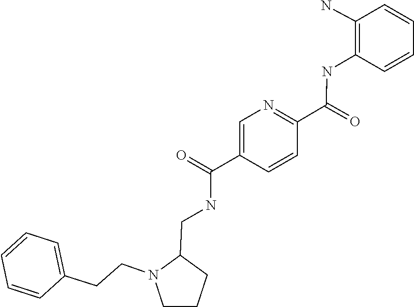 | 1039 |
| 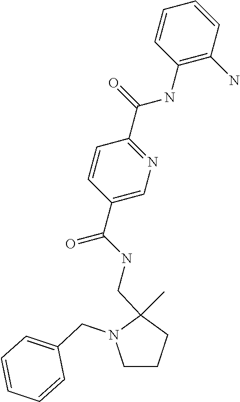 [SMILES: CC1(CNC(=O)c2ccc(C(=O)Nc3ccccc3N)nc2)CCCN1Cc1ccccc1] | 1040 |
| 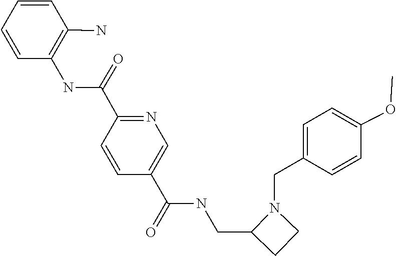 | 1041 |
| 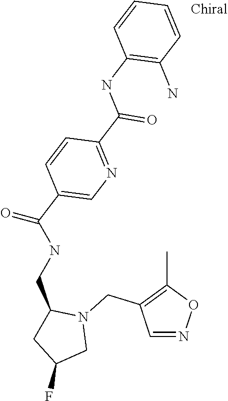 [SMILES: Cc1oncc1CN1C[C@@H](F)C[C@H]1CNC(=O)c1ccc(C(=O)Nc2ccccc2N)nc1] | 1042 |
| 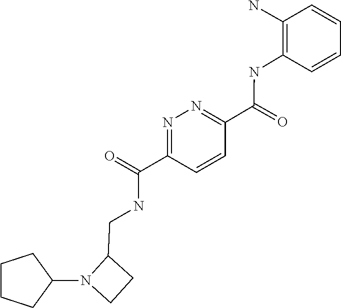 | 1043 |
| 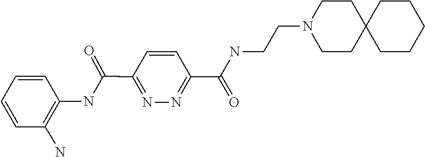 | 1044 |
| 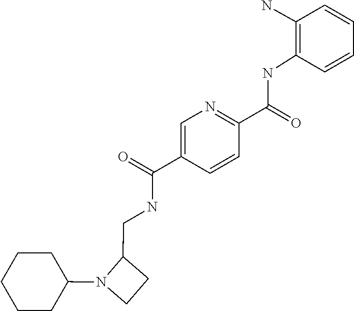 | 1045 |
| 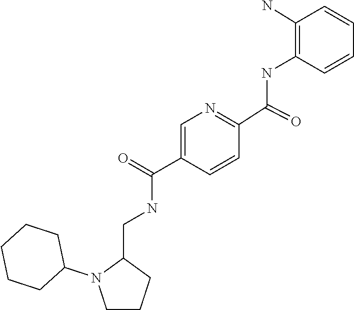 | 1046 |
| 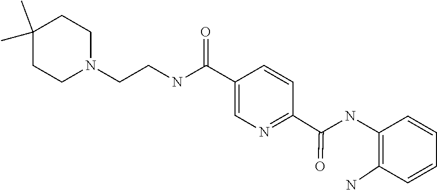 | 1047 |
| 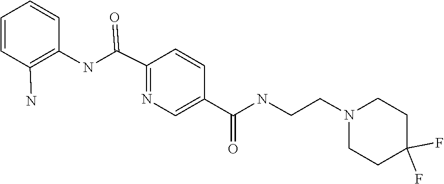 | 1048 |
| 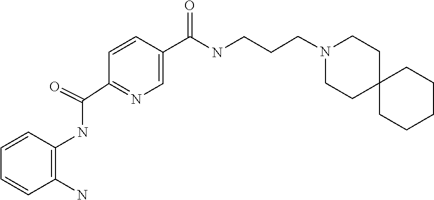 | 1049 |
| 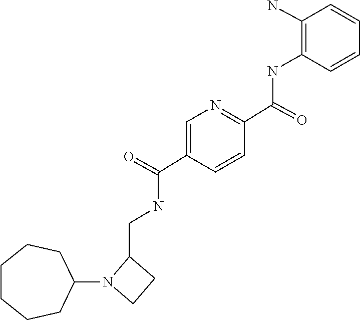 | 1050 |
| 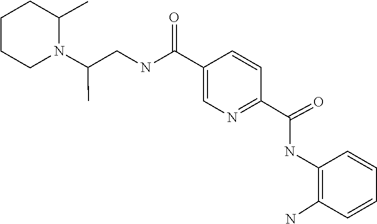 | 1051 |
| 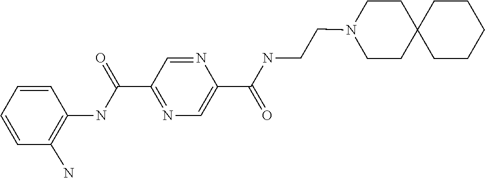 | 1052 |
| 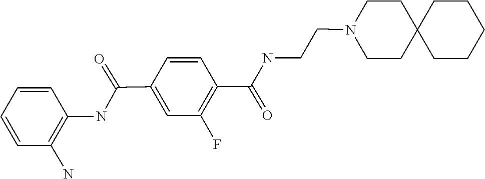 | 1053 |
| 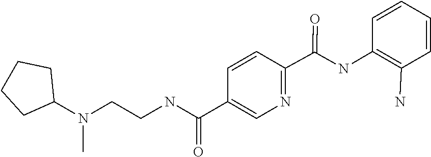 | 1054 |
| 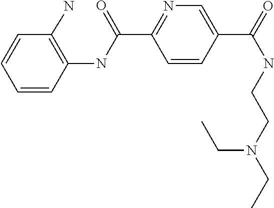 | 1055 |
| 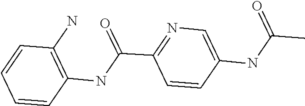 | 1056 |
| 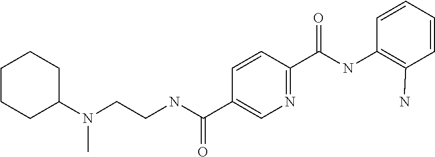 | 1057 |
| 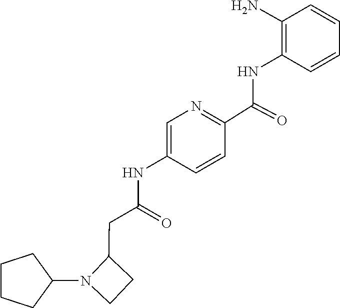 [SMILES: Nc1ccccc1NC(=O)c1ccc(NC(=O)CC2CCN2C2CCCC2)cn1] | 1058 |
| 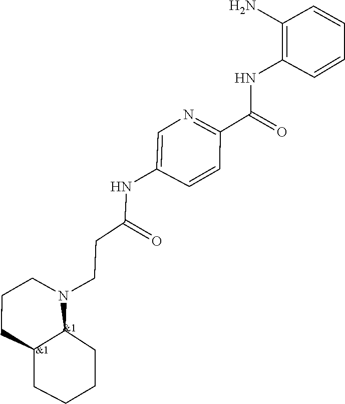 [SMILES: Nc1ccccc1NC(=O)c1ccc(NC(=O)CCN2CCC[C@@H]3CCCC[C@@H]32)cn1] | 1059 |
| 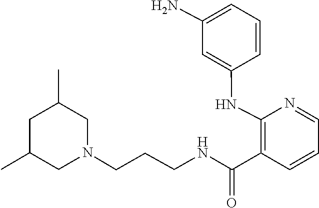 [SMILES: CC1CC(C)CN(CCCNC(=O)c2cccnc2Nc2cccc(N)c2)C1] | 1060 |
| 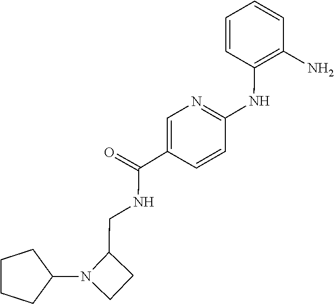 [SMILES: Nc1ccccc1Nc1ccc(C(=O)NCC2CCN2C2CCCC2)cn1] | 1061 |
| 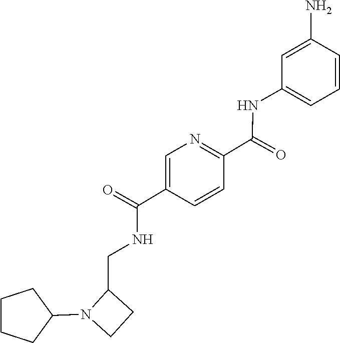 [SMILES: Nc1cccc(NC(=O)c2ccc(C(=O)NCC3CCN3C3CCCC3)cn2)c1] | 1062 |
| 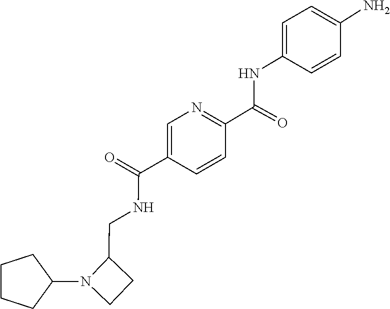 [SMILES: Nc1ccc(NC(=O)c2ccc(C(=O)NCC3CCN3C3CCCC3)cn2)cc1] | 1063 |
| 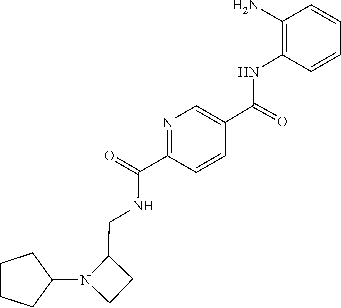 [SMILES: Nc1ccccc1NC(=O)c1ccc(C(=O)NCC2CCN2C2CCCC2)nc1] | 1064 |
| 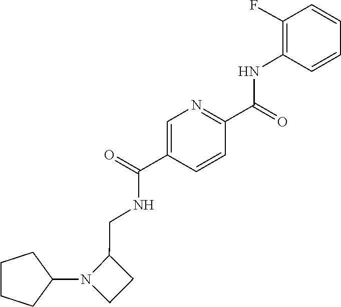 [SMILES: O=C(NCC1CCN1C1CCCC1)c1ccc(C(=O)Nc2ccccc2F)nc1] | 1065 |
| 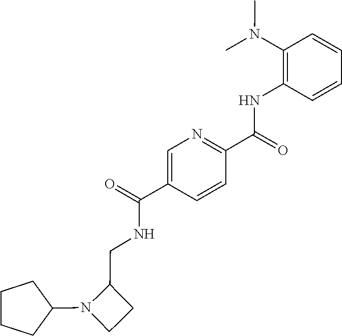 [SMILES: CN(C)c1ccccc1NC(=O)c1ccc(C(=O)NCC2CCN2C2CCCC2)cn1] | 1066 |
| 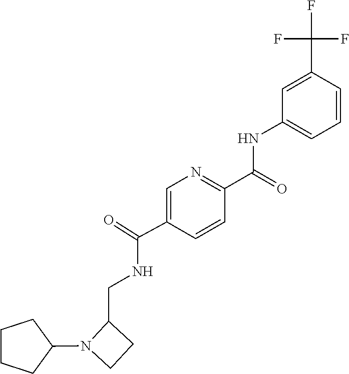 [SMILES: O=C(NCC1CCN1C1CCCC1)c1ccc(C(=O)Nc2cccc(C(F)(F)F)c2)nc1] | 1067 |
| 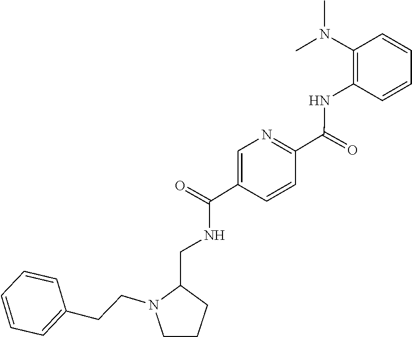 [SMILES: CN(C)c1ccccc1NC(=O)c1ccc(C(=O)NCC2CCCN2CCc2ccccc2)cn1] | 1068 |
| 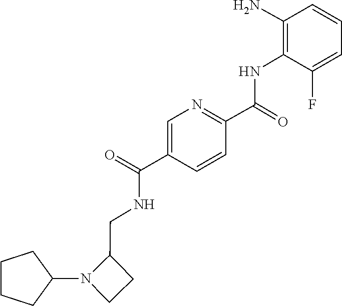 [SMILES: Nc1cccc(F)c1NC(=O)c1ccc(C(=O)NCC2CCN2C2CCCC2)cn1] | 1069 |
| 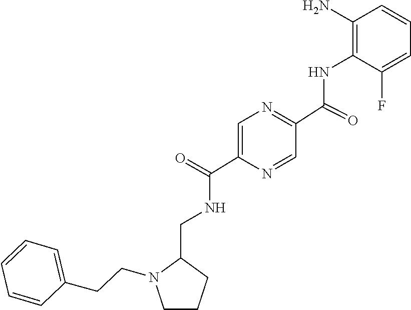 [SMILES: Nc1cccc(F)c1NC(=O)c1cnc(C(=O)NCC2CCCN2CCc2ccccc2)cn1] | 1070 |

In some cases, the compound of Table A is 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, or 1057, or a pharmaceutically acceptable salt thereof.

In some cases, a compound for use in disclosed methods is as shown in Table B, or a pharmaceutically acceptable salt thereof.

TABLE B

| Compound | ID |
|---|---|
| 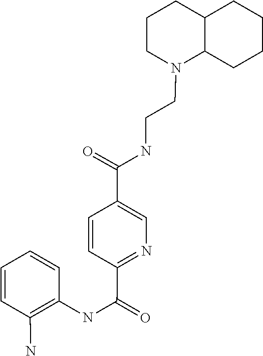 | 1009 |
| 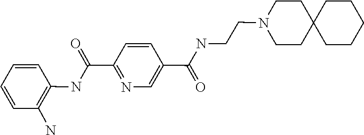 | 1013 |
| 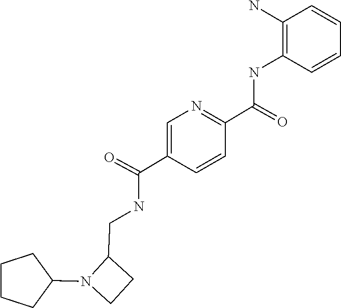 | 1015 |
| 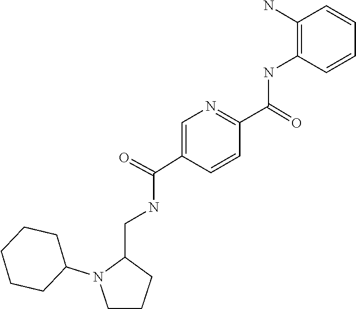 | 1046 |
| 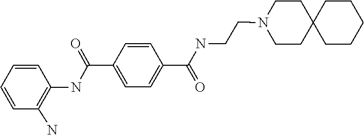 | 1020 |
| 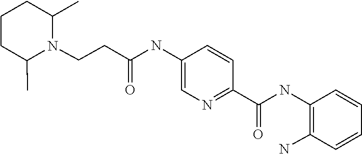 | 1023 |
| 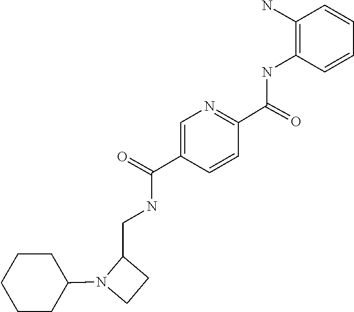 | 1045 |
| 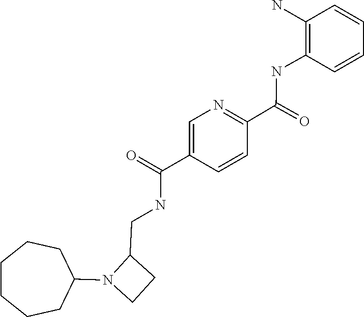 | 1050 |

In some cases, the compound for use in the disclosed methods is one of Compounds 1009, 1011, 1013, 1015, 1020, 1045, 1046, 1050, 1023, 1038, 1039, or 1041, or a pharmaceutically acceptable salt thereof.

In some cases, the compound for use in the disclosed methods is one of Compounds 1054, 1055, 1056, or 1057, or a pharmaceutically acceptable salt thereof.

The salts, e.g., pharmaceutically acceptable salts, of compounds disclosed herein can be prepared by reacting the appropriate base or acid with an appropriate amount of compound.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include anions, for example sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, and mandelate.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N [symbol] dibenzylethylenediamine, chloroprocaine, choline, diethanolamine, dicyclohexylamine, ethylenediamine, N-methylglucamine, and procaine.

The compounds can be synthesized as disclosed in, e.g., International Application No. PCT/US2020/41186.

### Dosing and Pharmaceutical Formulations

Dosages of the compounds disclosed herein can be administered as a dose measured in mg/kg. Contemplated mg/kg doses of the disclosed compounds include about 0.001 mg/kg to about 1000 mg/kg. Specific ranges of doses in mg/kg include about 0.1 mg/kg to about 500 mg/kg, about 0.5 mg/kg to about 200 mg/kg, about 1 mg/kg to about 100 mg/kg, about 1 mg/kg to about 50 mg/kg, about 1 mg/kg to about 40 mg/kg, and about 5 mg/kg to about 30 mg/kg.

A compound used in a method described herein can be administered in an amount of about 0.005 to about 750 milligrams per dose, about 0.05 to about 500 milligrams per dose, or about 0.5 to about 250 milligrams per dose. For example, a compound can be administered, per dose, in an amount of about 0.005, 0.05, 0.5, 1, 2, 3, 4, 5, 10, 15, 20, 30, 40, 50, 100, 150, 200, 250, 300, 350, 400, 450, 500, 550, 600, 650, 700, or750 milligrams, including all doses between 0.005 and 750 milligrams.

As herein, the compounds described herein may be formulated in pharmaceutical compositions with a pharmaceutically acceptable excipient, carrier, or diluent. The compound or composition comprising the compound is administered by any route that permits treatment of the disease or condition. One route of administration is oral administration. Additionally, the compound or composition comprising the compound may be delivered to a patient using any standard route of administration, including parenterally, such as intravenously, intraperitoneally, intrapulmonary, subcutaneously or intramuscularly, intrathecally, topically, transdermally, rectally, orally, nasally or by inhalation. Slow release formulations may also be prepared from the agents described herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

Administration may take the form of single dose administration, or a compound as disclosed herein can be administered over a period of time, either in divided doses or in a continuous-release formulation or administration method (e.g., a pump). However the compounds disclosed herein are administered to the subject, the amounts of compound administered and the route of administration chosen should be selected to permit efficacious treatment of the disease or condition.

In an embodiment, the pharmaceutical compositions are formulated with one or more pharmaceutically acceptable excipient, such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, the pH is adjusted to a range from about pH 5.0 to about pH 8. More particularly, the pharmaceutical compositions may comprise a therapeutically effective amount of at least one compound as described herein, together with one or more pharmaceutically acceptable excipients. Optionally, the pharmaceutical compositions may comprise a combination of the compounds described herein, or may include a second active ingredient useful in the treatment or prevention of a disorder as disclosed herein.

Formulations, e.g., for parenteral or oral administration, are most typically solids, liquid solutions, emulsions or suspensions, while inhalable formulations for pulmonary administration are generally liquids or powders. A pharmaceutical composition can also be formulated as a lyophilized solid that is reconstituted with a physiologically compatible solvent prior to administration. Alternative pharmaceutical compositions may be formulated as syrups, creams, ointments, tablets, and the like.

The term “pharmaceutically acceptable excipient” refers to an excipient for administration of a pharmaceutical agent, such as the compounds described herein. The term refers to any pharmaceutical excipient that may be administered without undue toxicity.

Pharmaceutically acceptable excipients are determined in part by the particular composition being administered, as well as by the particular method used to administer the composition. Accordingly, there exists a wide variety of suitable formulations of pharmaceutical compositions (see, e.g., Remington [symbol] Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The pharmaceutical compositions described herein are formulated in any form suitable for an intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules wherein the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents (e.g., sodium carboxymethylcellulose, methylcellulose, hydroxypropyl methylcellulose, sodium alginate, polyvinylpyrrolidone, gum tragacanth, gum acacia); dispersing or wetting agents (e.g., a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate)); and thickening agents (e.g., carbomer, beeswax, hard paraffin or cetyl alcohol). The suspensions may also contain one or more preservatives (e.g., acetic acid, methyl or n-propyl p-hydroxy-benzoate); one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions may be in the form of a sterile injectable preparation, such as a sterile injectable aqueous emulsion or oleaginous suspension. This emulsion or suspension may be formulated by a person of ordinary skill in the art using those suitable dispersing or wetting agents and suspending agents, including those mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer [symbol] solution, and isotonic sodium chloride solution. In addition, sterile fixed oils may be employed as a solvent or suspending medium. For this purpose any bland fixed oil may be employed including synthetic mono- or diglycerides. In addition, fatty acids (e.g., oleic acid) may likewise be used in the preparation of injectables.

To obtain a stable water-soluble dose form of a pharmaceutical composition, a pharmaceutically acceptable salt of a compound described herein may be dissolved in an aqueous solution of an organic or inorganic acid, such as 0.3 M solution of succinic acid, or more preferably, citric acid. If a soluble salt form is not available, the compound may be dissolved in a suitable co-solvent or combination of co-solvents. Examples of suitable co-solvents include alcohol, propylene glycol, polyethylene glycol 300, polysorbate 80, glycerin and the like in concentrations ranging from about 0 to about 60% of the total volume. In one embodiment, the active compound is dissolved in DMSO and diluted with water.

The pharmaceutical composition may also be in the form of a solution of a salt form of the active ingredient in an appropriate aqueous vehicle, such as water or isotonic saline or dextrose solution. Also contemplated are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In some embodiments, the compounds described herein may be formulated for oral administration in a lipid-based formulation suitable for low solubility compounds. Lipid-based formulations can generally enhance the oral bioavailability of such compounds.

As such, pharmaceutical compositions comprise a therapeutically or prophylactically effective amount of a compound described herein, together with at least one pharmaceutically acceptable excipient selected from the group consisting of medium chain fatty acids and propylene glycol esters thereof (e.g., propylene glycol esters of edible fatty acids, such as caprylic and capric fatty acids) and pharmaceutically acceptable surfactants, such as polyoxyl 40 hydrogenated castor oil.

### Embodiments

1. A method of treating a central nervous system (CNS) injury in a subject comprising administering to the subject a therapeutically effective amount of a compound as listed in Table A, or B, or having a structure of Formula (I):

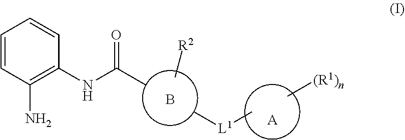

wherein
ring B-R² is

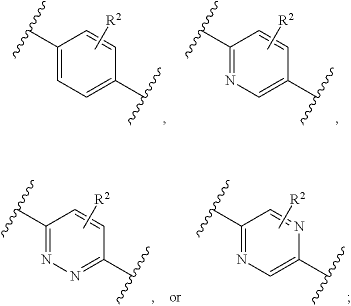

L¹ is NH—CO—C₀₋₃alkylene or CO—NH—C₀₋₄alkylene;
ring A is a 4-12-membered monocyclic, bicyclic, bridged, or spiro heterocycle comprising a nitrogen ring atom;
each R¹ is independently H, C₁₋₆alkyl, halo, C₁₋₆haloalkyl, C₁₋₃alkylene-O—C₁₋₃alkyl, C₀₋₃alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl;
R² is H, F, OH, OMe, or NH₂; and
n is 1, 2, or 3,
   with the proviso that when ring A comprises piperidinyl, at least one R¹ is other than H, or a pharmaceutically acceptable salt thereof.
2. The method of embodiment 1, wherein ring B is

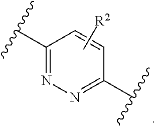

3. The method of embodiment 1, wherein ring B is

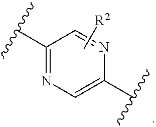

4. The method of embodiment 1, wherein ring B is

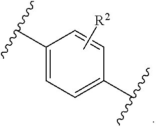

5. The method of embodiment 1, wherein ring B is

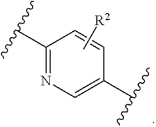

6. The method of embodiment 1, wherein the compound has a structure of Formula (IA), (IA′), (IA″), (IA″′), (IB), (IB′), (IC), or (IC′):

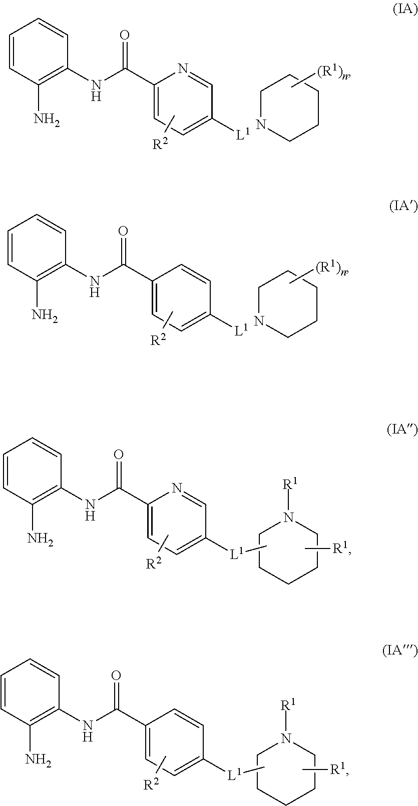

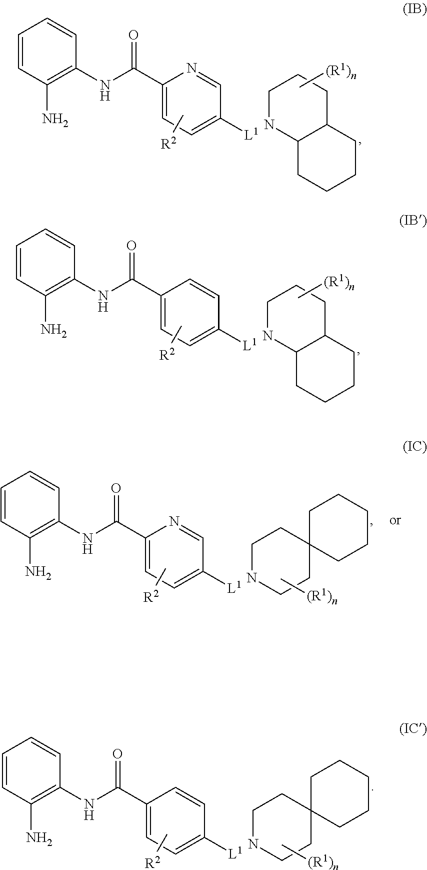

7. The method of embodiment 1, wherein the compound has a structure of Formula (ID) or (ID′):

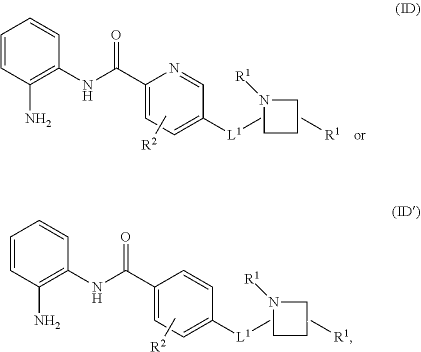

wherein when L¹ is attached to the ring nitrogen, R¹ on the ring nitrogen is null.
8. The method of embodiment 1, wherein the compound has a structure of Formula (IE) or (IE′):

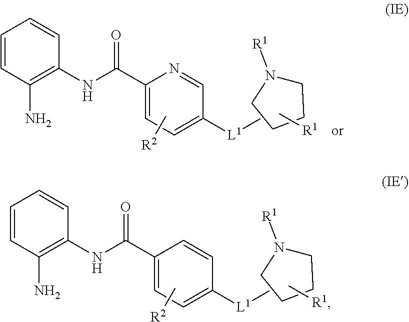

wherein when L¹ is attached to the ring nitrogen, R¹ on the ring nitrogen is null.
9. The method of any one of embodiments 1 to 5, wherein ring A comprises azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl.
10. The method of any one of embodiments 1 to 9, wherein at least one R¹ is fluoro, cyclopropyl, cyclopentyl, cyclohexyl, cycloheptyl, CH₂cyclohexyl, CH₂cyclopenyl, methyl, CH₂CH₂OCH₃, isopropyl, difluoroethyl, trifluoroethyl, tetrahydropyranyl, CH₂-(methyl-isoxazolyl), methyl-pyrazolyl, CH₂CH₂phenyl, CH₂phenyl, CH₂(methoxyphenyl), or phenyl.
11. The method of any one of embodiments 1 to 10, wherein L¹ is NHCO—CO—C₀₋₄alkylene.
12. The method of any one of embodiments 1 to 10, wherein L¹ is CONH—CO—C₀₋₄alkylene.
13. The method of embodiment 12, wherein Lis CONHCH₂.
14. The method of embodiment 12, wherein L¹ is CONHCH₂CH₂.
15. The method of embodiment 12, wherein L¹ is CONHCH₂CH(CH₃).
16. The method of any one of embodiments 1 to 15, wherein R² is F.
17. The method of any one of embodiments 1 to 15, wherein R² is H.
18. The method of any one of embodiments 1 to 15, wherein R² is NH₂, OMe, or OH.
19. The method of embodiment 1, wherein the compound, or pharmaceutically acceptable salt thereof, is listed in Table A.
20. The method of embodiment 1, wherein the compound, or pharmaceutically acceptable salt thereof, is listed in Table B.
21. The method of embodiment 1, wherein the compound has a structure of

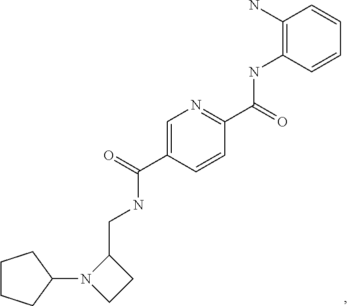 [SMILES: Nc1ccccc1NC(=O)c1ccc(C(=O)NCC2CCN2C2CCCC2)cn1]

or a pharmaceutically acceptable salt thereof.
22. The method of any one of embodiments 1 to 21, wherein the CNS injury is a stroke.
23. The method of embodiment 22, wherein the stroke is an ischemic stroke.
24. The method of embodiment 22, wherein the stroke is a hemorrhagic stroke.
25. The method of any one of embodiments 1 to 21, wherein the CNS injury is a traumatic brain injury.
26. The method of any one of embodiments 1 to 21, wherein the CNS injury is a spinal cord injury.
27. The method of any one of embodiments 1 to 21, wherein the CNS injury is a cerebral small vessel disease.

### Examples

### Example 1—Wnt Modulator Activates Wnt/β-Catenin Signaling in Neuronal N2a Cells

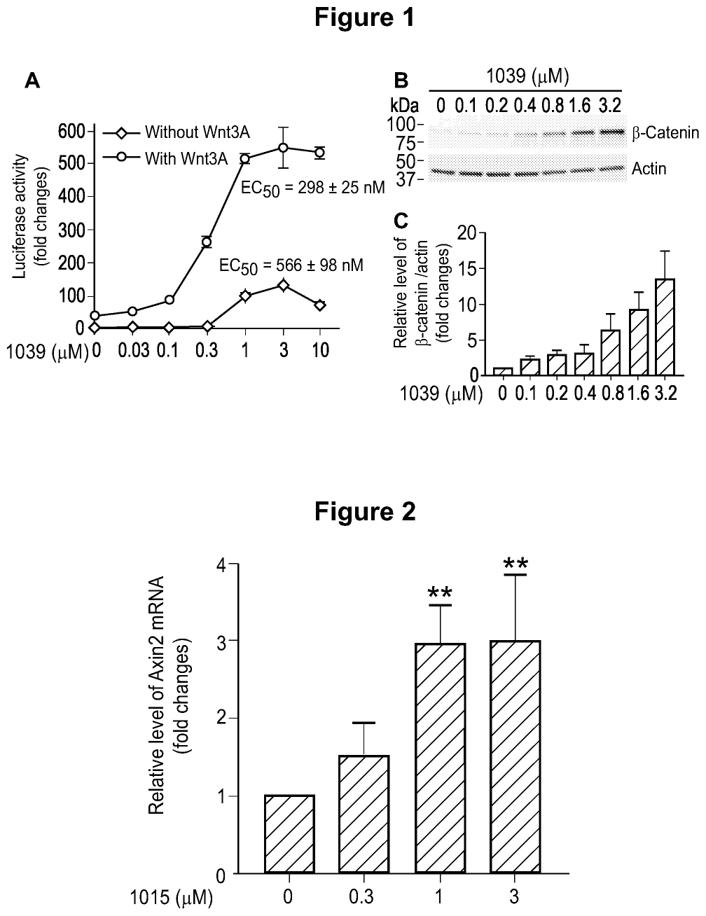

Wnt/β-catenin signaling plays a critical role in neuronal survival, neurogenesis, synaptogenesis, neuronal plasticity and synaptic plasticity [3-5]. While Wnt/β-catenin signaling is greatly suppressed in central nervous system injuries, restoring Wnt/β-catenin signaling represents a unique opportunity for the rational design of treatment for central nervous system injuries. FIG. 1A shows that Compound 1039 displays a potent activity in enhancing Wnt reporter Super8XTOPFlash activity in Wnt3A-expressing HEK293 cells with an EC₅₀ value at 298±25 nm (X±SE). Moreover, Compound 1039 enhances β-catenin level in a dose dependent manner in neuronal N2a cells (FIGS. 1B & 1C).

### Example 2: Wnt Modulator Activates Wnt/β-Catenin Signaling in Human Cerebral Microvascular Endothelial Cells

The blood-brain barrier (BBB) is a highly selective semipermeable border of endothelial cells that protects CNS neurons from exposure to neurotoxic blood-derived debris, cells and microbial pathogens. Studies in past ten years have established that the Wnt/β-catenin pathway is a key pathway required not only for BBB formation but also for BBB integrity and function [3]. Moreover, BBB breakdown following middle cerebral artery occlusion can be potently rescued in mice in which Wnt/β-catenin is specifically activated in brain endothelial cells [29]. On the other hand, dysregulation of Wnt/β-catenin may limit remyelination of white matter lesions (WMLs) and render the BBB leaky [24, 27, 28]. Compound 1015 is able to activate Wnt/β-catenin signaling in hCMEC/D3 human cerebral microvascular endothelial cells (FIG. 2), suggesting that Compound 1015 has a therapeutic potential in rescuing BBB breakdown and promoting post-stroke angiogenesis.

### Example 3: Neuroprotection and Reduction of the Infarct of Wnt Modulators in Mouse Stroke & Brain Ischemia Models

The effect of Wnt modulators in neuroprotection and reduction of the infarct in animals is tested using middle cerebral artery occlusion (MCAO) in mice. This model involves the insertion of a surgical filament into the external carotid artery and threading it forward into the internal carotid artery (ICA) until the tip occludes the origin of the MCA, resulting in a cessation of blood flow and subsequent brain infarction in the middle cerebral artery territory [30]. Therefore, C57BL6 mice with distal MCAO at 2-3 months of age are given the Wnt modulator daily via intraperitoneal injection (IP) or oral gavage (PO) at 5 mg/kg to 50 mg/kg, 5 injections per week, for 3 weeks. After treatment, the mice are examined by behavioral tests, and then sacrificed for biological analyses on neuronal cell death, neurogenesis, angiogenesis, neuroinflammation, BBB integrity and permeability, and infarct volume.

### Example 4: Neuroprotection of Wnt Modulators in Early Brain Injury in Rat Subarachnoid Hemorrhage Models

The effect of Wnt modulators in neuroprotection in animals is tested using subarachnoid hemorrhage (SHA) in rat models. Experimental subarachnoid hemorrhage rat models are induced by injecting autologous blood into the prechiasmatic cistern [31]. Wnt modulator interventions are applied once SAH models are established. Wnt modulators are given daily via intraperitoneal injection (IP) at 5 mg/kg to 50 mg/kg for 2 days. Brain tissue samples are obtained at 48 hours in the different groups, and cortical cell apoptosis, neuroinflammation, brain edema and neurological impairment are examined.

### Example 5: Neuroprotection of Wnt Modulators In Mouse Traumatic Brain Injury (TBI) Models

The effect of Wnt modulators in neuroprotection in animals is tested using controlled cortical impact (CCI) of TBI model in mice. The CCI model is one of the most commonly used models of pre-clinical TBI, and the deficits caused by CCI models have been found to mimic neurobehavioral and cognitive deficits, as they are typically seen after human TBI. CCI in mice is induced with a PCI3000 precision cortical impactor (Hatteras Instruments, Cary, NC) and a 2.8-mm diameter impact tip (velocity=3.0 m/sec, depth=0.5 mm, and contact duration=150 msec) [17]. Wnt modulator interventions are applied 2 hours post-TBI, and then are given daily via intraperitoneal injection (IP) or oral gavage (PO) at 5 mg/kg to 50 mg/kg, 5 injections per week, for 4 weeks. After treatment, the mice are examined by behavioral tests on depression, anxiety, impairment of spatial learning and memory, and multiple motor deficits. The mice are then sacrificed for biological analyses on neuronal cell death, neurogenesis and neuroinflammation.

### Example 6: Neuroprotection of Wnt Modulators in Rat Spinal Cord Injury Models

The effect of Wnt modulators in neuroprotection in animals is tested using contusion model of spinal cord injury (SCI) in rats. The contusion model is established with the modified weight-drop method in rats at 11-13 weeks old and weighing between 220 and 250 g as previously described [32, 33]. Wnt modulator interventions are applied immediately after SCI, and then are given daily via intraperitoneal injection (IP) or oral gavage (PO) at 5 mg/kg to 50 mg/kg, 5 injections per week, for 4 weeks. Behavioral assessment is performed prior to SCI at 24 and 72 h after SCI and then weekly for 6 weeks. Specifically, locomotor function is graded using the Basso-Beattie-Bresnahan (BBB) locomotor rating scale [34], and functional recovery of motor locomotion activity is used to evaluate animals [symbol] aibility to maintain their body position on an inclined plate [35]. At the end experiment, the rats are then sacrificed for biological analyses on motor neuronal survival, neurogenesis, neuroinflammation, BBB integrity and permeability, angiogenesis, and lesion size after SCI.

### REFERENCES

[1] C. Niehrs, The complex world of WNT receptor signalling, Nat Rev Mol Cell Biol, 13 (2012) 767-779.
[2] R. Nusse, H. Clevers, Wnt/beta-Catenin Signaling, Disease, and Emerging Therapeutic Modalities, Cell, 169 (2017) 985-999.
[3] L. Jia, J. Pina-Crespo, Y. H. Li, Restoring Wnt/beta-catenin signaling is a promising therapeutic strategy for Alzheimer [symbol] disease, Mol Brain, 12 (2019).
[4] R. Menet, S. Lecordier, A. ElAli, Wnt Pathway: An Emerging Player in Vascular and Traumatic Mediated Brain Injuries, Front Physiol, 11 (2020).
[5] T. Knotek, L. Janeckova, J. Kriska, V. Korinek, M. Anderova, Glia and Neural Stem and Progenitor Cells of the Healthy and Ischemic Brain: The Workplace for the Wnt Signaling Pathway, Genes-Basel, 11 (2020).
[6] Z. G. Zhang, M. Chopp, Neurorestorative therapies for stroke: underlying mechanisms and translation to the clinic, Lancet Neurol, 8 (2009) 491-500.
[7] C. Lambert, P. Cisternas, N. C. Inestrosa, Role of Wnt Signaling in Central Nervous System Injury, Mol Neurobiol, 53 (2016) 2297-2311.
[8] F. Mastroiacovo, C. L. Busceti, F. Biagioni, S. G. Moyanova, M. H. Meisler, G. Battaglia, A. Caricasole, V. Bruno, F. Nicoletti, Induction of the Wnt antagonist, Dickkopf-1, contributes to the development of neuronal death in models of brain focal ischemia, J Cerebr Blood F Met, 29 (2009) 264-276.
[9] T. Seifert-Held, T. Pekar, T. Gattringer, N. E. Simmet, H. Scharnagl, T. Stojakovic, F. Fazekas, M. K. Storch, Circulating Dickkopf-1 in acute ischemic stroke and clinically stable cerebrovascular disease, Atherosclerosis, 218 (2011) 233-237.
[10] X. W. He, E. Wang, Y. Y. Bao, F. Wang, M. Zhu, X. F. Hu, X. P. Jin, High serum levels of sclerostin and Dickkopf-1 are associated with acute ischaemic stroke, Atherosclerosis, 253 (2016) 22-28.
[11] Z. Z. Wei, J. Y. Zhang, T. M. Taylor, X. Gu, Y. Zhao, L. Wei, Neuroprotective and regenerative roles of intranasal Wnt-3a administration after focal ischemic stroke in mice, J Cereb Blood Flow Metab, 38 (2018) 404-421.
[12] N. Matei, J. Camara, D. McBride, R. Camara, N. Xu, J. Tang, J. H. Zhang, Intranasal wnt3a Attenuates Neuronal Apoptosis through Frz1/PIWIL1a/FOXM1 Pathway in MCAO Rats, J Neurosci, 38 (2018) 6787-6801.
[13] Y. Wang, D. J. Bao, B. Xu, C. D. Cheng, Y. F. Dong, X. P. Wei, C. S. Niu, Neuroprotection mediated by the Wnt/Frizzled signaling pathway in early brain injury induced by subarachnoid hemorrhage, Neural Regen Res, 14 (2019) 1013-1024.
[14] W. Ruan, J. Hu, H. Zhou, Y. Li, C. Xu, Y. Luo, T. Chen, B. Xu, F. Yan, G. Chen, Intranasal wnt-3a alleviates neuronal apoptosis in early brain injury post subarachnoid hemorrhage via the regulation of wnt target PPAN mediated by the moonlighting role of aldolase C, Neurochem Int, 134 (2020) 104656.
[15] K. Blennow, D. L. Brody, P. M. Kochanek, H. Levin, A. McKee, G. M. Ribbers, K. Yaffe, H. Zetterberg, Traumatic brain injuries, Nat Rev Dis Primers, 2 (2016) 16084.
[16] X. Ke, M. Yang, J. M. Luo, Y. Zhang, X. Y. Chen, The role of serum Dickkopf-1 in predicting 30-day death in severe traumatic brain injury, Brain Behav, 10 (2020) e01589.
[17] J. Y. Zhang, J. H. Lee, X. Gu, Z. Z. Wei, M. J. Harris, S. P. Yu, L. Wei, Intranasally Delivered Wnt3a Improves Functional Recovery after Traumatic Brain Injury by Modulating Autophagic, Apoptotic, and Regenerative Pathways in the Mouse Brain, J Neurotrauma, 35 (2018) 802-813.
[18] C. Y. Chang, M. Z. Liang, C. C. Wu, P. Y. Huang, H. I. Chen, S. F. Yet, J. W. Tsai, C. F. Kao, L. Y. Chen, WNT3A Promotes Neuronal Regeneration upon Traumatic Brain Injury, Int J Mol Sci, 21 (2020).
[19] N. A. Silva, N. Sousa, R. L. Reis, A. J. Salgado, From basics to clinical: a comprehensive review on spinal cord injury, Prog Neurobiol, 114 (2014) 25-57.
[20] J. M. Yu, J. H. Kim, G. S. Song, J. S. Jung, Increase in proliferation and differentiation of neural progenitor cells isolated from postnatal and adult mice brain by Wnt-3a and Wnt-5a, Mol Cell Biochem, 288 (2006) 17-28.
[21] J. H. Park, J. Min, S. R. Baek, S. W. Kim, I. K. Kwon, S. R. Jeon, Enhanced neuroregenerative effects by scaffold for the treatment of a rat spinal cord injury with Wnt3a-secreting fibroblasts, Acta Neurochir (Wien), 155 (2013) 809-816.
[22] H. I. Suh, J. Min, K. H. Choi, S. W. Kim, K. S. Kim, S. R. Jeon, Axonal regeneration effects of Wnt3a-secreting fibroblast transplantation in spinal cord-injured rats, Acta Neurochir (Wien), 153 (2011) 1003-1010.
[23] M. Mustapha, C. M. N. C. M. Nesse, N. Aminuddin, A. A. Safri, M. M. Ghazali, Cerebral Small Vessel Disease (CSVD)—Lessons From the Animal Models, Front Physiol, 10 (2019).
[24] N. Manukjan, Z. Ahmed, D. Fulton, W. M. Blankesteijn, S. Foulquier, A Systematic Review of WNT Signaling in Endothelial Cell Oligodendrocyte Interactions: Potential Relevance to Cerebral Small Vessel Disease, Cells, 9 (2020).
[25] B. Engelhardt, S. Liebner, Novel insights into the development and maintenance of the blood-brain barrier, Cell Tissue Res, 355 (2014) 687-699.
[26] S. Liebner, R. M. Dijkhuizen, Y. Reiss, K. H. Plate, D. Agalliu, G. Constantin, Functional morphology of the blood-brain barrier in health and disease, Acta Neuropathol, 135 (2018) 311-336.
[27] L. Wang, J. Geng, M. Qu, F. Yuan, Y. Wang, J. Pan, Y. Li, Y. Ma, P. Zhou, Z. Zhang, G. Y. Yang, Oligodendrocyte precursor cells transplantation protects blood-brain barrier in a mouse model of brain ischemia via Wnt/beta-catenin signaling, Cell Death Dis, 11 (2020) 9.
[28] T. J. Yuen, J. C. Silbereis, A. Griveau, S. M. Chang, R. Daneman, S. P. J. Fancy, H. Zahed, E. Maltepe, D. H. Rowitch, Oligodendrocyte-encoded HIF function couples postnatal myelination and white matter angiogenesis, Cell, 158 (2014) 383-396.
[29] J. L. Chang, M. R. Mancuso, C. Maier, X. B. Liang, K. Yuki, L. Yang, J. W. Kwong, J. Wang, V. R. M. Vallon, M. Vallon, C. Kosinski, J. J. H. Zhang, A. T. Mah, L. J. Xu, L. Li, S. Gholamin, T. F. Reyes, R. Li, F. Kuhnert, X. Y. Han, J. Yuan, S. H. Chiou, A. D. Brettman, L. Daly, D. C. Corney, S. H. Cheshier, L. D. Shortliffe, X. W. Wu, M. Snyder, P. Chan, R. G. Giffard, H. Y. Chang, K. Andreasson, C. J. Kuo, Gpr124 is essential for blood-brain barrier integrity in central nervous system disease, Nat Med, 23 (2017) 450-+.
[30] T. Chiang, R. O. Messing, W. H. Chou, Mouse model of middle cerebral artery occlusion, J Vis Exp, D0110.3791/2761(2011).
[31] Y. Wang, A. Gao, X. Xu, B. Dang, W. You, H. Li, Z. Yu, G. Chen, The Neuroprotection of Lysosomotropic Agents in Experimental Subarachnoid Hemorrhage Probably Involving the Apoptosis Pathway Triggering by Cathepsins via Chelating Intralysosomal Iron, Mol Neurobiol, 52 (2015) 64-77.
[32] A. Yacoub, M. C. Hajec, R. Stanger, W. Wan, H. Young, B. E. Mathern, Neuroprotective effects of perflurocarbon (oxycyte) after contusive spinal cord injury, J Neurotrauma, 31 (2014) 256-267.
[33] K. Gao, Z. Shen, Y. Yuan, D. Han, C. Song, Y. Guo, X. Mei, Simvastatin inhibits neural cell apoptosis and promotes locomotor recovery via activation of Wnt/beta-catenin signaling pathway after spinal cord injury, J Neurochem, 138 (2016) 139-149.
[34] D. M. Basso, M. S. Beattie, J. C. Bresnahan, A sensitive and reliable locomotor rating scale for open field testing in rats, J Neurotrauma, 12 (1995) 1-21.
[35] A. S. Rivlin, C. H. Tator, Regional spinal cord blood flow in rats after severe cord trauma, J Neurosurg, 49 (1978) 844-853.

What is claimed:

1. A method of treating a central nervous system (CNS) injury in a subject comprising administering to the subject a therapeutically effective amount of a compound as listed in Table A, or B, or having a structure of Formula (I): 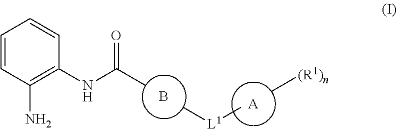
wherein
ring B-R² is 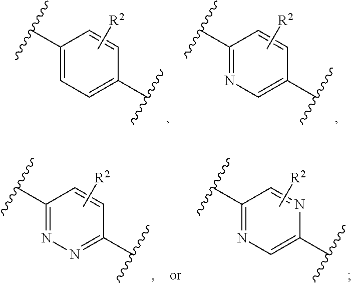
L¹ is NH—CO—C₀₋₃alkylene or CO—NH—C₀₋₄alkylene;
ring A is a 4-12-membered monocyclic, bicyclic, bridged, or spiro heterocycle comprising a nitrogen ring atom;
each R¹ is independently H, C₁₋₆alkyl, halo, C₁₋₆haloalkyl, C₁₋₃alkylene-O—C₁₋₃alkyl, C₀₋₅alkylene-C₃-C₈carbocycle, C₀₋₃alkylene-3-8-membered heterocycle, C₀₋₃alkylene-5-7-membered heteroaryl, or C₀₋₃alkylene-C₆₋₁₀aryl;
R² is H, F, OH, OMe, or NH₂; and
n is 1, 2, or 3,
with the proviso that when ring A comprises piperidinyl, at least one R¹ is other than H, or a pharmaceutically acceptable salt thereof.

2. The method of claim 1, wherein ring B is

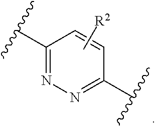

3. The method of claim 1, wherein ring B is

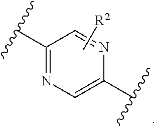

4. The method of claim 1, wherein ring B is

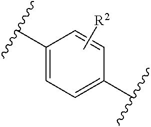

5. The method of claim 1, wherein ring B is

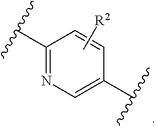

6. The method of claim 1, wherein the compound has a structure of Formula (IA), (IA′), (IA″), (IA″′), (IB), (IB′), (IC), or (IC′):

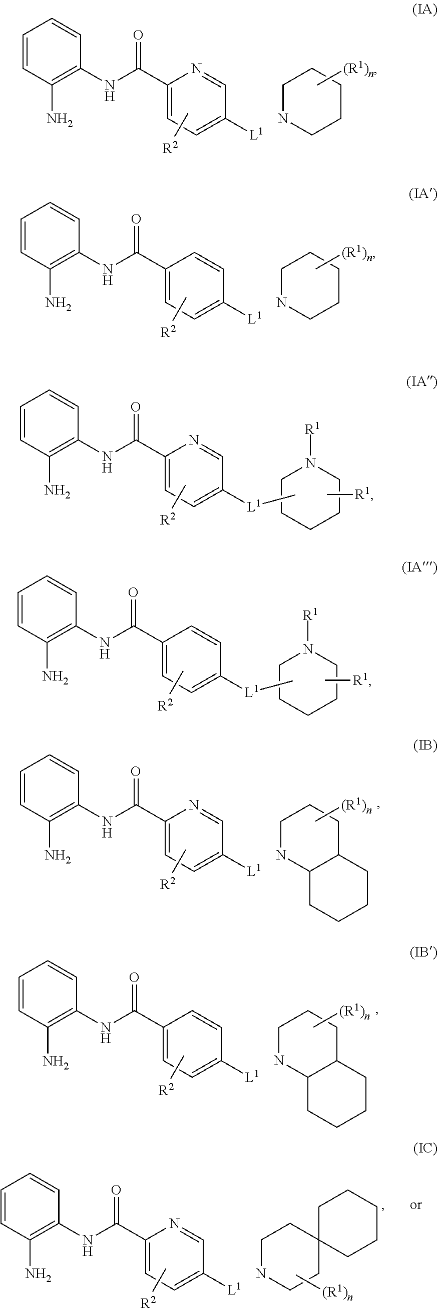

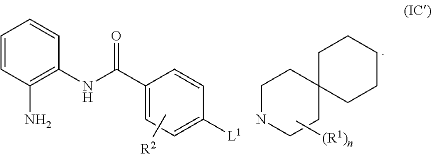

7. The method of claim 1, wherein the compound has a structure of Formula (ID) or (ID′): 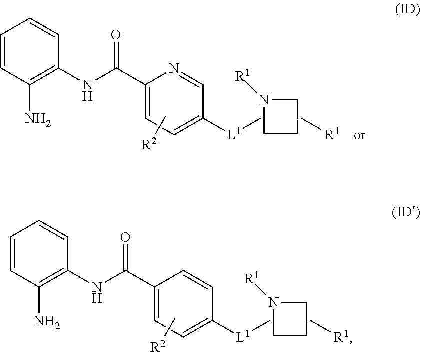
wherein when L¹ is attached to the ring nitrogen, R¹ on the ring nitrogen is null.

8. The method of claim 1, wherein the compound has a structure of Formula (IE) or (IE′): 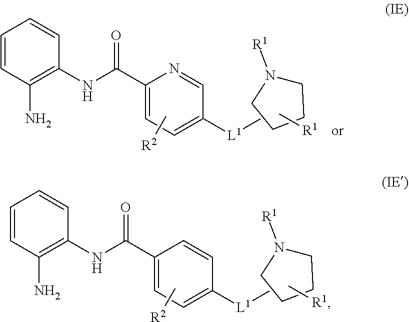
wherein when L¹ is attached to the ring nitrogen, R¹ on the ring nitrogen is null.

9. The method of any one of claims 1 to 5, wherein ring A comprises azetidinyl, piperidinyl, pyrollidinyl, decahydroquinolinyl, octahydroindolizinyl, quinuclidinyl, azaspiro[5.5]undecanyl, azabicyclo[2.1.1]hexanyl, azepanyl, or hexahydropyrrolizinyl.

10. The method of any one of claims 1 to 9, wherein at least one R¹ is fluoro, cyclopropyl, cyclopentyl, cyclohexyl, cycloheptyl, CH₂cyclohexyl, CH₂cyclopenyl, methyl, CH₂CH₂OCH₃, isopropyl, difluoroethyl, trifluoroethyl, tetrahydropyranyl, CH₂-(methyl-isoxazolyl), methyl-pyrazolyl, CH₂CH₂phenyl, CH₂phenyl, CH₂(methoxyphenyl), or phenyl.

11. The method of any one of claims 1 to 10, wherein L¹ is NHCO—C₀₋₄alkylene.

12. The method of any one of claims 1 to 10, wherein L¹ is CONH—C₀₋₄alkylene.

13. The method of claim 12, wherein Lis CONHCH₂.

14. The method of claim 12, wherein L¹ is CONHCH₂CH₂.

15. The method of claim 12, wherein L¹ is CONHCH₂CH(CH₃).

16. The method of any one of claims 1 to 15, wherein R² is F.

17. The method of any one of claims 1 to 15, wherein R² is H.

18. The method of any one of claims 1 to 15, wherein R² is NH₂, OMe, or OH.

19. The method of claim 1, wherein the compound, or pharmaceutically acceptable salt thereof, is listed in Table A.

20. The method of claim 1, wherein the compound, or pharmaceutically acceptable salt thereof, is listed in Table B.

21. The method of claim 1, wherein the compound has a structure of 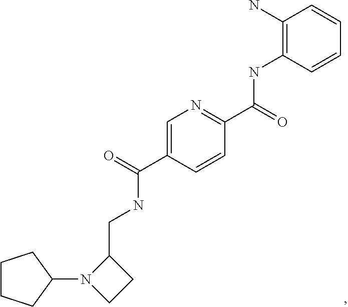 [SMILES: Nc1ccccc1NC(=O)c1ccc(C(=O)NCC2CCN2C2CCCC2)cn1]
or a pharmaceutically acceptable salt thereof.

22. The method of any one of claims 1 to 21, wherein the CNS injury is a stroke.

23. The method of claim 22, wherein the stroke is an ischemic stroke.

24. The method of claim 22, wherein the stroke is a hemorrhagic stroke.

25. The method of any one of claims 1 to 21, wherein the CNS injury is a traumatic brain injury.

26. The method of any one of claims 1 to 21, wherein the CNS injury is a spinal cord injury.

27. The method of any one of claims 1 to 21, wherein the CNS injury is a cerebral small vessel disease.
